(19) United States
(12) Patent Application Publication
(10) Pub. No.: US 2014/0186869 A1
(43) Pub. Date: Jul. 3, 2014

(54) METHOD FOR INHIBITING PROTEASE IN BIOLOGICAL SAMPLE CONTAINING PANCREATIC JUICE COMPONENTS

(71) Applicant: OLYMPUS CORPORATION, Tokyo (JP)

(72) Inventors: Rie KATAOKA, Tokyo (JP); Mari NAKATA, Tokyo (JP); Nao MORIYA, Tokyo (JP)

(73) Assignee: OLYMPUS CORPORATION, Tokyo (JP)

(21) Appl. No.: 14/200,188

(22) Filed: Mar. 7, 2014

Related U.S. Application Data
(63) Continuation of application No. PCT/JP2012/066004, filed on Jun. 22, 2012.

(30) Foreign Application Priority Data
Sep. 13, 2011 (JP) 2011-199357

(51) Int. Cl. C12Q 1/37

(52) U.S. Cl. CPC C12Q 1/37
USPC 435/23; 564/440

(57) ABSTRACT

This method for inhibiting protease in a biological sample containing pancreatic juice components inhibits protease enzyme activity in the biological sample by adding at least one type of protease inhibitor having a sulfonyl fluoride group to the biological sample containing pancreatic juice components. In addition, this protease inhibitor for a biological sample containing pancreatic juice components is a compound having a sulfonyl fluoride group, has protease inhibitory activity, and is added to a biological sample containing pancreatic juice components in order to inhibit protease present in the biological sample. Moreover, this kit for preserving a biological sample containing pancreatic juice components contains at least one type of protease inhibitory having a sulfonyl fluoride group, and is used to store a biological sample containing pancreatic juice components.

### BACKGROUND OF THE INVENTION

1. Field of the Invention

The present invention relates to a method for inhibiting protease enzyme activity in a biological sample containing pancreatic juice components, a protease inhibitor effective for suppressing the degradation of pancreatic juice-derived components in pancreatic juice or duodenal juice, and a kit for preserving biological samples such as pancreatic juice that contains the protease inhibitor.

The present application claims priority on the basis of Japanese Patent Application No. 2011-199357 filed in Japan on Sep. 13, 2011, the contents of which are incorporated herein by reference. The present application is a U.S. continuation application based on the PCT International Patent Application, PCT/JP2012/066004, filed on Jun. 22, 2012: the contents of which are incorporated herein by reference.

2. Description of the Related Art

Bodily fluids serve as important biological samples for determining the states of organs. Pancreatic juice is also an important biological sample for determining the state of the pancreas, and is used in cytodiagnosis, bicarbonate assay, bacteriological examinations and examinations using markers composed of proteins, nucleic acids and the like. Wide-ranging searches for novel markers for use in pancreatic cancer and other pancreas-related diseases are being made in research fields as well.

Since pancreatic juice is a digestive juice, it contains various digestive enzymes. Although these digestive enzymes are present in an inactive state in the pancreas, they are known to be activated after having been discharged into the duodenum. Digestive enzymes in pancreatic juice undergo a degradatory cascade reaction induced by intestinal enterokinase secreted from duodenal epithelial cells. Trypsinogen contained in pancreatic juice is activated by intestinal enterokinase resulting in conversion to trypsin, and this trypsin is known to trigger activation of various digestive enzymes (proteases) such as chymotrypsinogen and proesterase. Furthermore, trypsinogen is said to account for roughly 12% of the total protein content of pancreatic juice (see, for example, Yokoyama, et al., Pancreas, 2002, Vol. 24, No. 4, pp. 344-347). As a result of activation of these various proteases, biomolecules such as proteins, nucleic acids, lipids or cells contained in pancreatic juice are degraded and denatured following discharge into the duodenum. Consequently, in the case of utilizing pancreatic juice discharged into the duodenum in testing and research such as cytodiagnosis or biomolecular assays, there is concern over measurements being unable to be performed accurately as a result of the target cells or proteins and the like present in pancreatic juice being affected by proteases. Consequently, it is important to inhibit the activity of proteases as much as possible in pancreatic juice discharged into the duodenum.

One example of a conventional method for reducing the activity of various degradatory enzymes present in pancreatic juice and duodenal juice consists of suppressing their activity by maintaining collected pancreatic juice in a chilled state so as to be outside the optimum temperature range of enzyme activity. In general, collected pancreatic juice and the like is required to immediately be placed in ice, and testing on that pancreatic juice and the like is required to be carried out promptly (see, for example, Katsunuma, et al., “Analysis”, 1978, Vol. 10, pp. 682-689). In this method, however, there is the potential for enzyme reactions to resume if the temperature of the pancreatic juice and the like rises. Consequently, it is necessary to constantly maintain the collected pancreatic juice at a low temperature, thereby making handling bothersome. In addition, since degradation proceeds even at low temperatures depending on the type of protein, simply maintaining at a low temperature is inadequate for suppressing degradation and denaturation of pancreatic juice-derived components.

In order to measure the protein in pancreatic juice, a method has been disclosed by which 0.2 mL of aprotinin is added to 0.5 mL of pancreatic juice collected with an endoscope followed by placing in frozen storage (see, for example, Yokoyama, et al., Pancreas, 2002, Vol. 24, No. 4, pp. 344-347). Nearly all protease activity in the pancreatic juice can be suppressed during the storage period by placing in frozen storage, thereby enabling cells or proteins and the like present in the pancreatic juice to be preserved in a stable state. However, since frozen pancreatic juice is required to be thawed prior to measurement, there are cases in which degradation of the cells or proteins resumes after thawing. Moreover, there is also the risk of the molecules targeted for analysis per se being damaged during frozen storage.

In addition, a method for preserving biological samples, and particularly blood, has been disclosed in which blood is collected directly into a container containing at least two types of protease inhibitors (see, for example, Japanese Patent No. 4496407). As a result of immediately contacting the collected blood with the protease inhibitors, degradation and the like of proteins in the blood can be prevented. In this method, a serine protease inhibitor and other protease inhibitors are used, and blood is collected into a container containing, for example, a serine protease inhibitor such as AEBSF, aprotinin or leupeptin, and a cysteine protease inhibitor such as E-64.

### SUMMARY OF THE INVENTION

As a result of conducting extensive studies to solve the aforementioned problems, the inventors of the present invention found that a protease inhibitor having a sulfonyl fluoride group demonstrates the highest activity inhibitory effects against proteases contained in pancreatic juice, thereby leading to completion of the present invention.

(1) A first aspect of the present invention is a method for inhibiting protease in a biological sample containing pancreatic juice components, comprising: adding at least one type of protease inhibitor having a sulfonyl fluoride group to the biological sample containing pancreatic juice components, thereby inhibiting protease enzyme activity in the biological sample.

(2) The method for inhibiting protease in a biological sample containing pancreatic juice components of (1) above, wherein the protease inhibitor having a sulfonyl fluoride group is one or more types of compounds selected from the group consisting of PMSF, AEBSF, p-APMSF, 4-(fluorosulfonyl)benzoic acid, 3-(fluorosulfonyl)benzoic acid, 2-aminobenzenesulfonyl fluoride, 3-aminobenzenesulfonyl fluoride, 4-aminobenzenesulfonyl fluoride, 2-nitrobenzenesulfonyl fluoride, 3-nitrobenzenesulfonyl fluoride and 4-nitrobenzenesulfonyl fluoride.

(3) The method for inhibiting protease in a biological sample containing pancreatic juice components of (1) above, wherein the protease inhibitor having a sulfonyl fluoride group is one or more types of compounds selected from the group consisting of PMSF, AEBSF and p-APMSF.

(4) The method for inhibiting protease in a biological sample containing pancreatic juice components of (1) above, wherein the protease inhibitor having a sulfonyl fluoride group is two or more types of compounds selected from the group consisting of PMSF, AEBSF and p-APMSF.

(5) The method for inhibiting protease in a biological sample containing pancreatic juice components of any of (1) to (4) above, wherein PMSF, AEBSF or p-APMSF is added to the biological sample so that the final concentration of PMSF is 1 mM or higher, the final concentration of AEBSF is 4 mM or higher, and the final concentration of p-APMSF is 2 mM or higher.

(6) The method for inhibiting protease in a biological sample containing pancreatic juice components of any of (1) to (5) above, wherein at least one type of protease inhibitor having a sulfonyl group is further added to the biological sample.

(7) The method for inhibiting protease in a biological sample containing pancreatic juice components of (6) above, wherein the protease inhibitor having a sulfonyl group is an amino acid chloromethyl ketone.

(8) The method for inhibiting protease in a biological sample containing pancreatic juice components of (6) above, wherein the protease inhibitor having a sulfonyl group is one or more types selected from the group consisting of TLCK and TPCK.

(9) The method for inhibiting protease in a biological sample containing pancreatic juice components of (6) above, wherein the protease inhibitor having a sulfonyl group is TLCK.

(10) The method for inhibiting protease in a biological sample containing pancreatic juice components of any of (1) to (9) above, wherein the biological sample is pancreatic juice or duodenal juice.

(11) A second aspect of the present invention is a protease inhibitor for a biological sample containing pancreatic juice components that is a compound having: a sulfonyl fluoride group, and protease inhibitory activity; and is added to a biological sample containing pancreatic juice components for inhibiting protease present in the biological sample.

(12) The protease inhibitor for a biological sample containing pancreatic juice components of (11) above is a compound selected from the group consisting of PMSF, AEBSF, p-APMSF, 4-(fluorosulfonyl)benzoic acid, 3-(fluorosulfonyl)benzoic acid, 2-aminobenzenesulfonyl fluoride, 3-aminobenzenesulfonyl fluoride, 4-aminobenzenesulfonyl fluoride, 2-nitrobenzenesulfonyl fluoride, 3-nitrobenzenesulfonyl fluoride and 4-nitrobenzenesulfonyl fluoride.

(13) The protease inhibitor for a biological sample containing pancreatic juice components of (11) above is a compound selected from the group consisting of PMSF, AEBSF and p-APMSF.

(14) A third aspect of the present invention is a protease inhibitor mixture comprising at least one type of the protease inhibitor for a biological sample of any of (11) to (13) above.

(15) The protease inhibitor mixture of (14) above further comprises at least one type of protease inhibitor having a sulfonyl group.

(16) A fourth aspect of the present invention is a kit for preserving a biological sample containing pancreatic juice components that comprises at least one type of protease inhibitory having a sulfonyl fluoride group, wherein the kit is used to store a biological sample containing pancreatic juice components.

(17) The kit for preserving a biological sample containing pancreatic juice components of (16) above further comprises at least one type of protease inhibitor having a sulfonyl group.

(18) The kit for preserving a biological sample containing pancreatic juice components of (16) or (17) above further comprises a storage container provided with a storage portion for storing collected body fluid, wherein the protease inhibitor having a sulfonyl fluoride group is preliminarily contained in the storage portion.

### BRIEF DESCRIPTION OF THE DRAWINGS

FIG. 1 is a drawing showing the chemical formulas of PMSF, AEBSF and p-APMSF.

FIG. 2 is a graph indicating fluorescence measured from various pancreatin solutions in Example 1.

FIG. 3 is a graph indicating the results of measuring S100P concentrations in various sample solutions in Example 2.

FIG. 4 is a graph indicating fluorescence measured from various sample solutions in Example 3.

FIG. 5 is a graph indicating fluorescence measured from various sample solutions in Example 4.

FIG. 6 is a graph indicating fluorescence measured from various sample solutions in Example 5.

FIG. 7 is a graph indicating fluorescence measured from various sample solutions in Example 5.

FIG. 8 is a graph indicating the results for protease activity (relative value: %) of various sample solutions in Example 6.

FIG. 9 is a graph indicating the results for protease activity (relative value: %) of various sample solutions to which PMSF was added in Example 7.

FIG. 10 is a graph indicating the results for protease activity (relative value: %) of various sample solutions to which AEBSF was added in Example 7.

FIG. 11 is a graph indicating the results for protease activity (relative value: %) of various sample solutions to which p-APMSF was added in Example 7.

FIG. 12 is a graph indicating fluorescence measured from various sample solutions to which one type of protease inhibitor was added in Example 8.

FIG. 13 is a graph indicating fluorescence measured from various sample solutions to which two types of protease inhibitors were added in Example 8.

FIG. 14 is a graph indicating fluorescence measured from various sample solutions to which various types of protease inhibitors were added in Example 9.

### DETAILED DESCRIPTION OF THE PREFERRED EMBODIMENTS

Pancreatic juice refers to a body fluid that is discharged from the pancreatic duct. In the present invention and description of the present application, a biological sample containing pancreatic juice components refers to a sample containing body fluid that contains components derived from pancreatic juice. Examples of body fluid that contains components derived from pancreatic juice include pancreatic juice collected directly from the pancreas with a catheter and liquid collected in the duodenum (duodenal juice). Duodenal juice contains pancreatic juice as well as bile similarly discharged from the papillary region, liquid inherently present in the duodenum, and blood. Pancreatic juice and duodenal juice can be collected by ordinary methods.

<Protease Inhibitor for Biological Sample Containing Pancreatic Juice Components>

The protease inhibitor for a biological sample containing pancreatic juice components according to the second aspect of the present invention (to also be referred to as the “protease inhibitor of the present invention”) is characterized as a compound that has a sulfonyl fluoride group, has protease inhibitory activity, and is added to a biological sample containing pancreatic juice components in order to inhibit protease present in the biological sample.

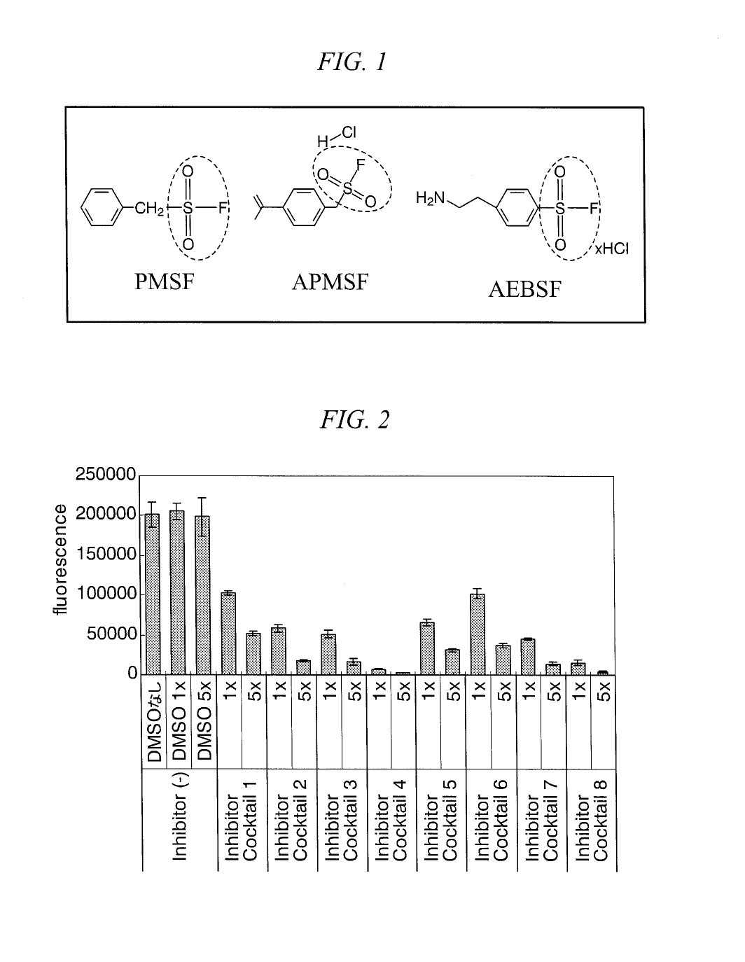

There are no particular limitations on the protease inhibitor of the present invention provided it is a compound that has a sulfonyl fluoride group and has protease inhibitory activity. It may be a compound having a structure in which a sulfonyl fluoride group is bound directly to a benzene ring or through a hydrocarbon group having 1 to 6 carbon atoms, it may be phenylmethylsulfonyl fluoride (PMSF), 4-(2-aminoethyl)-benzenesulfonyl fluoride (AEBSF), p-amidinophenylmethane sulfonyl fluoride hydrochloride (p-APMSF), 4-(fluorosulfonyl)benzoic acid (CAS No. 455-26-5), 3-(fluorosulfonyl)benzoic acid (CAS No. 454-95-5), 2-aminobenzenesulfonyl fluoride (CAS No. 392-86-9), 3-aminobenzenesulfonyl fluoride (CAS No. 368-50-3), 4-aminobenzenesulfonyl fluoride (CAS No. 98-62-4), 2-nitrobenzenesulfonyl fluoride (CAS No. 433-98-7), 3-nitrobenzenesulfonyl fluoride (CAS No. 349-78-0) or 4-nitrobenzenesulfonyl fluoride (CAS No. 349-96-2). It may be PMSF, AEBSF or p-APMSF. FIG. 1 shows the chemical formulas of PMSF, AEBSF and p-APMSF. In FIG. 1, the functional groups encircled by dotted lines are sulfonyl fluoride groups.

The protease inhibitor of the present invention may be an active ingredient of a mixture composed of a plurality of types of protease inhibitors. The protease inhibitor mixture may be composed only of the protease inhibitor of the present invention or may be a mixture of one type or two or more types of the protease inhibitor of the present invention and other protease inhibitors. There are no particular limitations on the other protease inhibitors provided they do not impair the protease inhibitory activity of the protease inhibitor of the present invention, and examples thereof include peptide-based protease inhibitors such as aprotinin, leupeptin, antipain, chymostatin, elastatinal and antithrombin; chelating agents such as EDTA; elastase inhibitor, TLCK, TPCK, trypsin inhibitor, ecotin and E. coli. In addition, pancreatitis therapeutic drugs such as gabexate mesilate (Foy), camostat mesilate (Foipan), nafamostat mesilate (Futhan) or ulinastatin can also be used.

Other protease inhibitors used in combination with the protease inhibitor of the present invention may consist of at least one type of protease inhibitor having a sulfonyl group, and it may consist of amino acid chloromethyl ketones. There are no particular limitations on the amino acid chloromethyl ketone provided it has protease inhibitory activity, and examples thereof include N-a-tosyl-L-lysine chloromethyl ketone (TLCK) and N-a-tosyl-L-phenylalanine chloromethyl ketone (TPCK). In the present invention, one or more types of amino acid chloromethyl ketones may be selected from the group consisting of TLCK and TPCK in particular, and it may be TLCK.

A mixture composed of a plurality of types of protease inhibitors, including the protease inhibitor of the present invention, may contain two or more types of the protease inhibitor of the present invention, it may contain two or more types of the protease inhibitor of the present invention and at least one type of protease inhibitor having a sulfonyl group, it may contain two or more types of the protease inhibitor of the present invention and at least one type of amino acid chloromethyl ketone having protease inhibitory activity, and particularly it may contain two or more types of compounds selected from the group consisting of PMSF, AEBSF and p-APMSF and one or more types of compounds selected from the group consisting of TLCK and TPCK. <Method for Inhibiting Protease in Biological Sample Containing Pancreatic Juice Components>

The method of inhibiting protease in a biological sample containing pancreatic juice components according to the first aspect of the present invention (to also be referred to as the “protease inhibition method of the present invention”) is characterized by adding at least one type of protease inhibitor having a sulfonyl fluoride group to the biological sample, thereby inhibiting protease enzyme activity in a biological sample containing pancreatic juice components. The protease inhibitor of the present invention has extremely high inhibitory activity against protease contained in pancreatic juice and duodenal juice in comparison with other protease inhibitors. Consequently, by adding the protease inhibitor of the present invention to a biological sample containing pancreatic juice components, degradation of pancreatic juice components by protease derived from pancreatic juice and duodenal juice present in the biological sample can be easily and effectively suppressed.

The biological sample containing pancreatic juice components used in the protease inhibition method of the present invention is a sample that contains body fluid containing components derived from pancreatic juice. For example, it may be composed only of body fluid containing components derived from pancreatic juice, a liquid obtained by diluting the body fluid with a suitable buffer and the like, or a liquid obtained by adding various types of additives to the body fluid or a diluted liquid thereof. Examples of additives include surfactants, nuclease inhibitors, pH adjusters and pH indicators. The biological sample containing pancreatic juice components used in the protease inhibition method of the present invention may be a sample that contains pancreatic juice or duodenal juice. Specific examples thereof include pancreatic juice, duodenal juice, diluted pancreatic juice or duodenal juice, and liquids obtained by adding the various types of additives listed above thereto.

In the protease inhibition method of the present invention, although the protease inhibitor of the present invention may be added to a biological sample containing pancreatic juice components that has been stored after having been collected from the body, the time interval from the time the body fluid containing pancreatic juice components is collected from the body until the time the protease inhibitor of the present invention is added thereto may be short, and the protease inhibitor of the present invention may be added to the pancreatic juice or duodenal juice immediately after having been collected from the body.

A wide variety of proteases are contained in pancreatic juice, examples of which include serine protease, metalloprotease and carboxypeptidase. Consequently, in the case of protease inhibitors conventionally used for pancreatic juice and the like, protease inhibitory effects obtained in the case of adding only one type of protease inhibitor are extremely limited, and even in the case of combining several types of protease inhibitors, the obtaining of adequate protease inhibitory effects has yet to be reported. In contrast, the protease inhibitor of the present invention allows the obtaining of adequate protease inhibitory effects by suitably adjusting the amount added even in the case of using only one type thereof.

Although adequate protease inhibitory activity is demonstrated even in the case of using only one type of the protease inhibitor of the present invention in the protease inhibition method of the present invention, two or more types may be used as combined. In addition, one type or two or more types of another protease inhibitor may be used together with the protease inhibitor of the present invention. The protease inhibitor of the present invention used in the protease inhibition method of the present invention may comprise one or more types of compounds selected from the group consisting of PMSF, AEBSF and p-APMSF, and it may comprise a combination of two or more types of compounds selected from the group consisting of PMSF, AEBSF and p-APMSF. Since pancreatic juice contains various proteases having different activities, protease activity in a biological sample can be expected to be more reliably suppressed by using a mixture of a plurality of protease inhibitors. Moreover, protease activity can be expected to be suppressed by combining at a lower concentration than in the case of using alone.

There are no particular limitations on the amount of protease inhibitor added to a biological sample containing pancreatic juice components provided it is an amount at which protease inhibitory effects are demonstrated by the protease inhibitor, and can be suitably adjusted in consideration of such factors as the type of biological sample containing pancreatic juice components or the type of protease inhibitor. For example, in the case of adding only PMSF as protease inhibitor to a biological sample containing pancreatic juice components, the PMSF is added so that the final concentration thereof may be 1 mM or higher, 5 mM or higher and 10 mM or higher. In addition, in the case of using only AEBSF as protease inhibitor, AEBSF can be added to a biological sample containing pancreatic juice components so that the final concentration thereof is 4 mM or higher, 10 mM or higher and 20 mM or higher. In the case of using only p-APMSF as protease inhibitor, it can be added to a biological sample containing pancreatic juice components so that the final concentration thereof is 2 mM or higher, 5 mM or higher and 10 mM or higher.

In addition, in the case of adding only TLCK as another protease inhibitor used together with at least one type of protease inhibitor having a sulfonyl fluoride group (protease inhibitor of the present invention), TLCK can be added so that the final concentration thereof is 0.1 mM or higher, 1 mM or higher, 5 mM or higher and 10 mM or higher. In addition, in the case of adding only TPCK as the other protease inhibitor, TPCK can be added so that the final concentration thereof is 0.1 mM or higher, 1 mM or higher, 5 mM or higher, and 10 mM or higher.

In the protease inhibition method of the present invention, the protease inhibitor of the present invention and other protease inhibitor added to a biological sample containing pancreatic juice components may be in the form of a solid such as a powder or granules, or may be a protease inhibitor solution obtained by dissolving in a suitable buffer and the like. In addition, the protease inhibitor of the present invention and other protease inhibitor may be added simultaneously to a biological sample containing pancreatic juice components, or one may be first added followed by adding the other. From the viewpoint of being able to adequately demonstrate inhibitory effects against protease present in a biological sample, the protease inhibitor having a sulfonyl fluoride group and the aforementioned other protease inhibitor may be added simultaneously.

Since the activity of proteases contained in a biological sample containing pancreatic juice components is effectively inhibited by adding the protease inhibitor of the present invention to the biological sample, degradation, denaturation and the like of pancreatic juice components contained in the biological sample are remarkably suppressed. Consequently, a biological sample containing pancreatic juice components in which protease has been inhibited by the protease inhibition method of the present invention allows biological components derived from pancreatic juice to be preserved more stably. In addition, analysis accuracy can be improved and highly reliable results can be obtained by analyzing pancreatic juice components obtained from biological samples containing pancreatic juice components in which protease has been inhibited by the protease inhibition method of the present invention.

A biological sample containing pancreatic juice components in which protease has been inhibited by the protease inhibition method of the present invention can be used as a measurement sample for various types of testing in the same manner as other biological samples. There are no particular limitations on the tested substance provided it is a biological component expected to be contained in pancreatic juice or duodenal juice, and it may be a protein, nucleic acid such as DNA and RNA, or cell. For example, the biological sample can be used in various types of protein analyses such as ELISA, immunochromatography, two-dimensional electrophoresis, western blotting or mass spectrometry, various types of nucleic acid analyses such as PCR, RT-PCR and probe-based hybridization, and various types of cytoanalyses in the manner of cell counting or cytodiagnosis.

<Kit for Preserving Biological Sample Containing Pancreatic Juice Components>

The kit for preserving a biological sample containing pancreatic juice components of the fourth aspect of the present invention (also referred to as the “preservation kit of the present invention”) contains at least one type of protease inhibitor having a sulfonyl fluoride group (namely, the protease inhibitor of the present invention), and is used to preserve biological samples containing pancreatic juice components. The use of this kit makes it possible to more easily inhibit protease present in a biological sample containing pancreatic juice components. Only one type of protease inhibitor of the present invention may be contained in the preservation kit of the present invention or a combination of two or more types may be contained. In addition, the kit may further contains at least one type of protease inhibitor having a sulfonyl group, and it may contain at least one type of amino acid chloromethyl ketone having protease inhibitory activity.

In addition, the protease inhibitor of the present invention contained in the preservation kit of the present invention may be in the form of a freeze-dried solid, may be in the form of a tablet or granules obtained by molding a freeze-dried powder with a suitable vehicle, or may be in the form of a protease inhibitor solution obtained by dissolving in a suitable buffer.

The preservation kit of the present invention may further contain a buffer for diluting a collected body fluid, other protease inhibitors, surfactant, pH adjuster or pH indicator and the like. Various types of additives such as a surfactant, pH adjuster or pH indicator may be preliminarily dissolved in a dilution buffer.

In addition, the preservation kit of the present invention may also contain a cap capable of joining to an opening of a container filled with a prepared sample containing pancreatic juice components, that allows a fixed amount of the sample (obtained by adding various types of protease inhibitors contained in the preservation kit of the present invention as well as other components as necessary to a biological sample containing pancreatic juice components) to be dropped from the container.

The preservation kit of the present invention may further contain a storage container provided with a storage portion for storing body fluid such as pancreatic juice or duodenal juice collected from the body. In this case, the protease inhibitor of the present invention and other protease inhibitors may be preliminarily contained in the storage portion. In the case graduations are formed in the storage container, the amount of biological sample added to the storage container (total amount of biological sample and protease inhibitor in the case protease inhibitor has been preliminarily filled into the storage container) can be confirmed visually, thereby enabling the final concentration of protease inhibitor to be roughly determined at a glance.

In addition, biological samples containing pancreatic juice components are typically collected transendoscopically. Therefore, a collection tool for collecting a biological sample containing pancreatic juice components transendoscopically may also be contained as a component of the preservation kit of the present invention. Examples of this collection tool include the combination of a syringe and catheter capable of being inserted into an endoscope apparatus, and a probe provided with an absorber on the end thereof capable of being inserted into an endoscope apparatus. An example of a catheter capable of being inserted into an endoscope apparatus is the specimen collection cube described in Japanese Unexamined Patent Application, First Publication No. 2011-5009. These collection tools may have the aforementioned storage portion preliminarily filled with protease inhibitor.

### EXAMPLES

Although the following provides a more detailed explanation of the present invention by indicating examples thereof, the present invention is not limited to the following examples.

### Example 1

A search was made of protease inhibitor cocktails that demonstrate highly effective inhibitory effects against proteases present in pancreatic juice from among eight types of commercially available protease inhibitor cocktails in order to determine the protease inhibitor having the greatest effect against proteases present in pancreatic juice. The protease inhibitor cocktails used consisted of Complete (Roche, Inhibitor Cocktail 1), Halt Protease Inhibitor Cocktail (Thermo, Inhibitor Cocktail 2), Protease Inhibitor Cocktail (Sigma, Inhibitor Cocktail 3), Protease Inhibitor Mix (GE, Inhibitor Cocktail 4), Protease Inhibitor Cocktail Set I (Merck, Inhibitor Cocktail 5), Protease Inhibitor Cocktail Set II (Merck, Inhibitor Cocktail 6), Protease Inhibitor Cocktail Set III (Merck, Inhibitor Cocktail 7), and Protease Inhibitor Cocktail (Biovision, Inhibitor Cocktail 8).

After adding each protease inhibitor cocktail so that the final concentration thereof was equal to manufacturer's recommended concentration (1×) or a concentration equal to 5 times the recommended concentration (5×), protease activity was measured using a simulated artificial pancreatic juice in the form of “pancreatin”, a digestive enzyme produced from porcine pancreas in which pancreatic enzymes are present in an activated state. Protease activity was measured using EnzCheck Protease Assay Kits (Molecular Probes). More specifically, fluorescent-labeled casein provided with the kits was added to a pancreatin solution to which each protease inhibitor cocktail had been added, and after incubating for 2 hours at 37° C., fluorescence was measured at a fluorescence wavelength of Ex/Em=485/535 nm. In addition, a sample solution in which fluorescent-labeled casein was added directly to pancreatin (inhibitor (−), DMSO (−)), a sample solution in which DMSO was added to pancreatin so that the added amount of DMSO was the same as in the case of adding each protease inhibitor cocktail at the manufacturer's recommended concentration (1×) (inhibitor (−), DMSO 1×), and a sample solution in which DMSO was added to pancreatin so that the added amount of DMSO was the same as in the case of adding each protease inhibitor cocktail at a concentration equal to five times the manufacturer's recommended concentration (5×) (inhibitor (−), DMSO 5×) were prepared for use as controls, and these sample solutions were measured for protease activity in the same manner as the protease inhibitor cocktails.

The results of measuring fluorescence of each pancreatin solution are shown in FIG. 2. Fluorescence plotted on the vertical axis represents the degraded amount of casein, and that value was estimated to be the value of protease activity. According to these results, Inhibitor Cocktail 4 (GE, Protease Inhibitor Mix) was determined to demonstrate the greatest inhibitory effect.

### Example 2

A study was conducted of the manner in which the storage stability of protein in pancreatic juice is affected by inhibition of protease activity. The degree to which S100P (a calcium-bound protein typically contained in pancreatic juice) is preserved in the case of adding or not adding protease inhibitor to a solution consisting of a mixture of S100P and pancreatin (simulated pancreatic juice) was investigated.

More specifically, a 25 ng/mL S100P (standard) solution (Sample Solution 1), a solution containing 25 ng/mL S100P (standard) and 1 mg/mL of pancreatin (Sample Solution 2), a solution containing 25 ng/mL of S100P (standard) and 1 mg/mL of pancreatin, and further containing Protease Inhibitor Mix (GE) at twice the recommended concentration (2×) (Sample Solution 3), and a solution containing 25 ng/mL of S100P (standard) and 1 mg/mL of pancreatin, and further containing Complete (Roche) at five times the recommended concentration (5×) (Sample Solution 4) were first prepared. Each sample solution was prepared using the buffer provided with the Circulex S100P ELISA Kit (Cyclex, Catalog No.: CY-8060). These sample solutions were allowed to react by incubating for 16 hours at 25° C. Subsequently, each sample solution was diluted 10-fold using the buffer provided with the Circulex S100P ELISA Kit (Cyclex, Catalog No.: CY-8060), and S100P was detected in the diluted sample solutions using the Circulex S100P ELISA Kit.

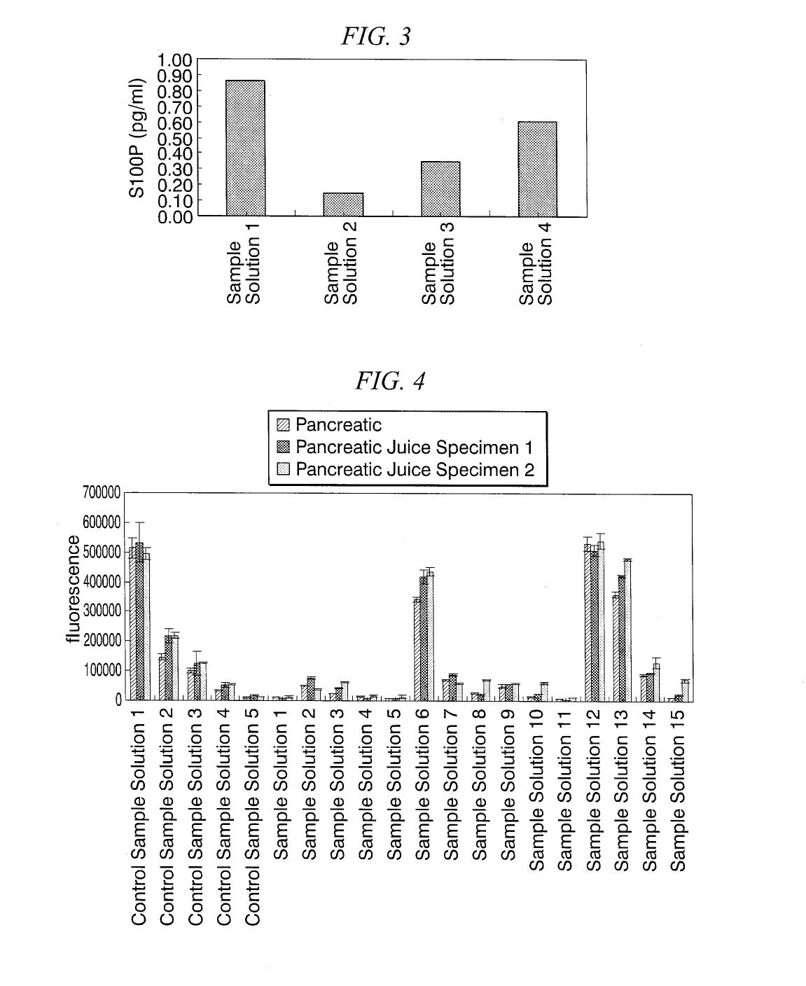

The results of measuring S100P in each sample solution are shown in FIG. 3. As a result, the residual amount of S100P was greater in Sample Solutions 3 and 4 to which protease inhibitor cocktail had been added than in Sample Solution 2 to which protease inhibitor cocktail had not been added. In addition, the residual amount of S100P was greater in Sample Solution 4, to which was added protease inhibitor cocktail having higher protease inhibitory activity than the protease inhibitor cocktail added to Sample Solution 3 (see Example 1), than in Sample Solution 3. On the basis of these results, the addition of protease inhibitor to pancreatic juice was clearly determined to suppress protein degradation in pancreatic juice and allow pancreatic juice to be preserved more stably. In addition, there was suggested to be a correlation between protease activity and detected concentration of S100P.

### Example 3

A study was conducted to determine which protease inhibitor demonstrates the highest level of inhibitory activity against protease present in pancreatic juice among various types of protease inhibitors contained in the inhibitor cocktail demonstrating the highest level of protease inhibitory activity in Example 1.

There are four types of serine protease inhibitors contained in Protease Inhibitor Mix (GE) consisting of aprotinin, leupeptin, PMSF and AEBSF (concentrations are not disclosed). Therefore, protease activity was measured in the same manner as Example 1 using pancreatin and two types of pancreatic juice specimens for 15 combinations of the four types of inhibitors prepared so that the final concentration was equal to the maximum recommended concentration for each protease inhibitor alone. The protease inhibitors added to each sample solution are shown in Table 1. Furthermore, the maximum recommended concentrations consisted of 0.3 μM for aprotinin (Roche), 50 μM for leupeptin (Roche), 1 mM for PMSF (Roche) and 4 mM for AEBSF (Roche). In addition, protease activity was also measured in the same manner for a sample solution to which protease inhibitor was not added (Control Sample Solution 1), a sample solution containing the recommended concentration of Inhibitor Cocktail 1 (Complete, Roche) (Control Sample Solution 2), a sample solution containing Inhibitor Cocktail 1 at 5 times the recommended concentration (5×) (Control Sample Solution 3), a sample solution containing the recommended concentration of Inhibitor Cocktail 4 (Protease Inhibitor Mix, GE) (Control Sample Solution 4), and a sample solution containing Inhibitor Cocktail 4 at five times the recommended concentration (5×) (Control Sample Solution 5).

TABLE 1

| | Aprotinin | Leupeptin | PMSF | AEBSF |
|---|---|---|---|---|
| Sample Solution 1 | ◯ | ◯ | ◯ | ◯ |
| Sample Solution 2 | ◯ | ◯ | ◯ | |
| Sample Solution 3 | ◯ | ◯ | | ◯ |
| Sample Solution 4 | ◯ | | ◯ | ◯ |
| Sample Solution 5 | | ◯ | ◯ | ◯ |
| Sample Solution 6 | ◯ | ◯ | | |
| Sample Solution 7 | ◯ | | ◯ | |
| Sample Solution 8 | ◯ | | | ◯ |
| Sample Solution 9 | | ◯ | ◯ | |
| Sample Solution 10 | | ◯ | | ◯ |
| Sample Solution 11 | | | ◯ | ◯ |
| Sample Solution 12 | ◯ | | | |
| Sample Solution 13 | | ◯ | | |
| Sample Solution 14 | | | ◯ | |
| Sample Solution 15 | | | | ◯ |

The results of measuring fluorescence of each sample solution are shown in FIG. 4. Fluorescence plotted on the vertical axis represents the degraded amount of casein, and that value was estimated to be the value of protease activity. Sample Solution 1 simulates Inhibitory Cocktail 4 and contains all four types of serine protease inhibitors, and hardly any degradation of casein was observed. Sample Solutions 4, 5 and 11 demonstrated protease inhibitory effects nearly equal to that of Sample Solution 1. The protease inhibitors added in common to these three types of sample solutions were PMSF and AEBSF. On the basis of these results, the combination of PMSF and AEBSF was clearly determined to demonstrate a high level of protease inhibitory effects in pancreatic juice. On the other hand, aprotinin and leupeptin, which are peptide-based protease inhibitors used in blood collection tubes as protease inhibitors for use in blood testing, had hardly any effect on protease activity.

### Example 4

PMSF and AEBSF are both compounds that have a sulfonyl fluoride group. Therefore, a study was conducted on the inhibitory effects of protease inhibitors having a sulfonyl fluoride group and protease inhibitors not having a sulfonyl fluoride group. PMSF (Roche), AEBSF (Roche), p-APMSF (Sigma), TLCK (Sigma) and TPCK (Sigma) were used as protease inhibitors.

More specifically, a sample solution obtained by adding 1 mM PMSF and 4 mM AEBSF, a sample solution obtained by adding 1 mM PMSF, a sample solution obtained by adding 4 mM AEBSF, a sample solution obtained by adding 5 mM p-APMSF, a sample solution obtained by adding 100 μl TLCK and a sample solution obtained by adding 100 μM TPCK were prepared for use as protease inhibitors, and the protease activity of each sample solution was measured in the same manner as Example 1 using pancreatin and two types of pancreatic juice specimens. In addition, the protease activities of a sample solution to which protease inhibitor was not added (inhibitor (−)), a sample solution containing Inhibitor Cocktail 1 at the recommended concentration (Complete, Roche), a sample solution containing Inhibitor Cocktail 1 at 5 times the recommended concentration (5×), a sample solution containing Inhibitor Cocktail 4 at the recommended concentration (Protease Inhibitor Mix, GE), and a sample solution containing Inhibitor Cocktail 4 at five times the recommended concentration (5×) were also measured in the same manner.

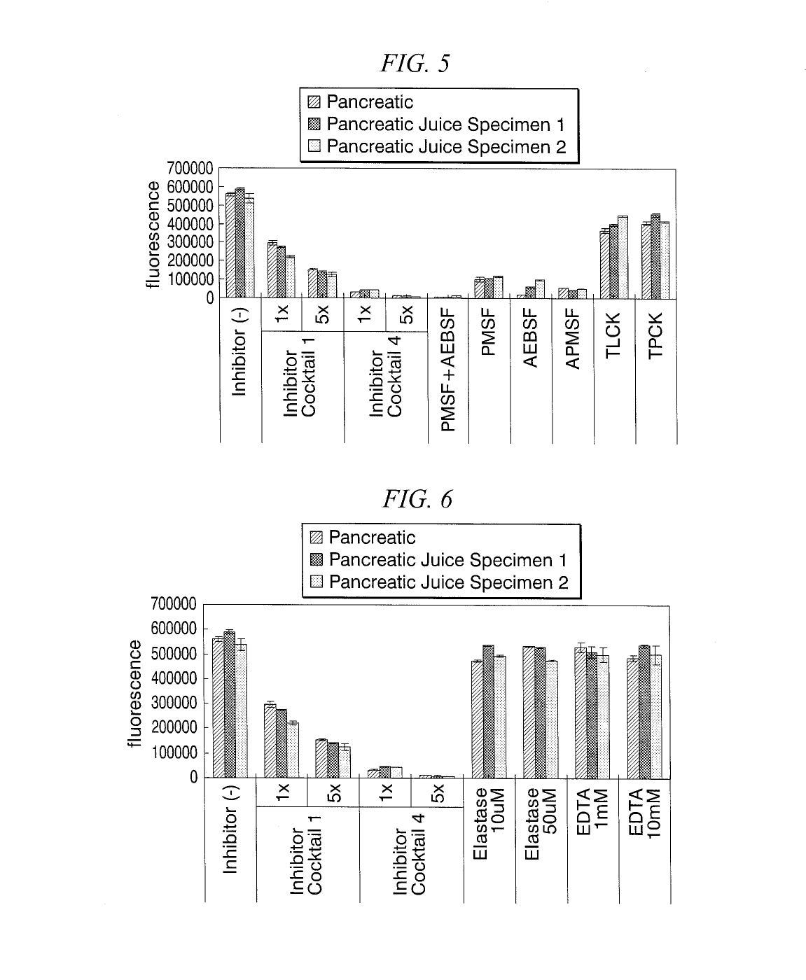

The results of measuring the fluorescence of each sample solution are shown in FIG. 5. Fluorescence plotted on the vertical axis represents the degraded amount of casein, and that value was estimated to be the value of protease activity. The protease inhibitors added to each sample solution are plotted on the horizontal axis. As a result, PMSF, AEBSF and p-APMSF demonstrated high levels of protease inhibitory effects, while TLCK and TPCK were clearly determined to demonstrate low levels of protease inhibitory effects against protease present in pancreatic juice.

As shown in FIG. 1, PMSF, AEBSF and p-APMSF all have chemical structures that are extremely similar. In particular, these compounds all have in common a sulfonyl fluoride group. Consequently, the possibility was suggested that the presence of a sulfonyl fluoride group has a significant effect on inhibition of protease in pancreatic juice. It is thought that a structure having a sulfonyl fluoride group also acts effectively on inhibition of serine proteases, which are known to trigger a degradatory cascade in pancreatic juice.

### Example 5

A study was conducted of the protease inhibitory effects of protease inhibitors not having a sulfonyl fluoride group. Elastase Inhibitor I (Merck), EDTA (Wako Pure Chemical Industries), Aprotinin (Roche), Leupeptin (Roche), TLCK (Sigma) and TPCK (Sigma) were used as protease inhibitors.

More specifically, a sample solution obtained by adding 10 μm to 50 μm Elastase Inhibitor I, a sample solution obtained by adding 1 mM to 10 mM EDTA, a sample solution obtained by adding 0.0003 mM to 0.3 mM Aprotinin, a sample solution obtained by adding 0.005 mM to 5 mM Leupeptin, a sample solution obtained by adding 0.01 mM to 1 mM TLCK or a sample solution obtained by adding 0.01 mM to 1 mM TPCK was prepared for use as protease inhibitor, and the protease activity of each sample solution was measured in the same manner as Example 1 using pancreatin and two types of pancreatic juice specimens. In addition, the protease activities of a sample solution to which protease inhibitor was not added (Inhibitor (−)), a sample solution containing Inhibitor Cocktail 1 at the recommended concentration (Complete, Roche), a sample solution containing Inhibitor Cocktail 1 at five times the recommended concentration (5×), a sample solution containing Inhibitor Cocktail 4 at the recommended concentration (Protease Inhibitor Mix, GE), and a sample solution containing Inhibitor Cocktail 4 at five times the recommended concentration (5×) were also measured in the same manner.

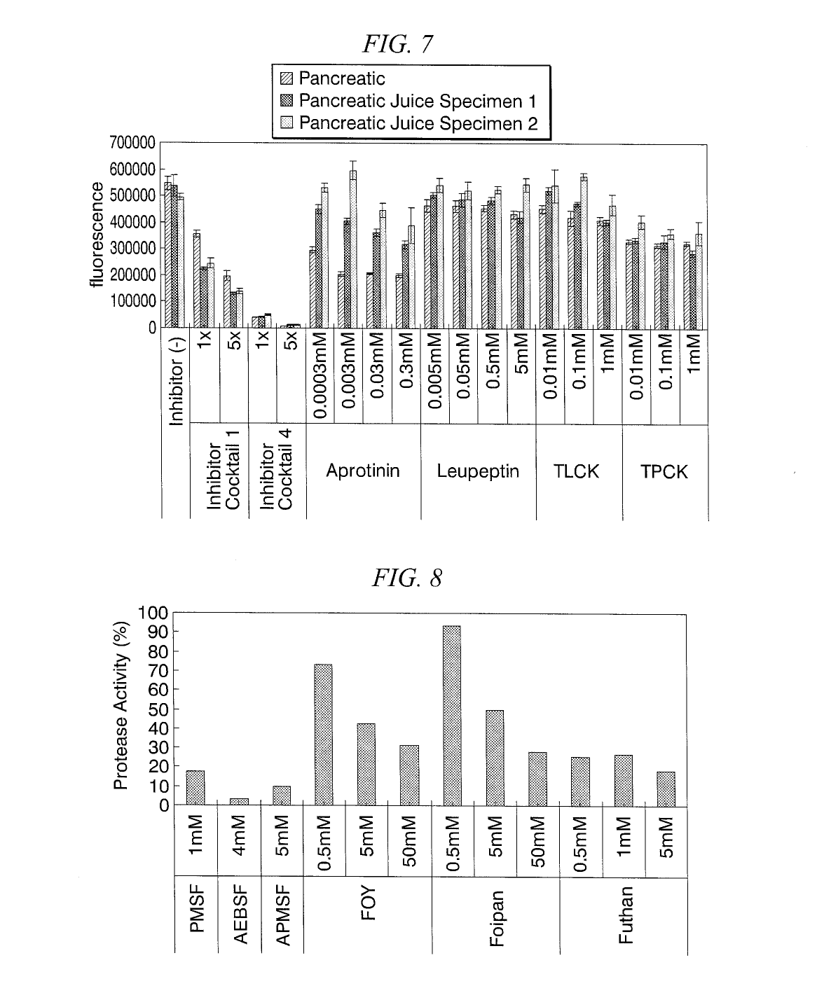

The results of measuring the fluorescence of each sample solution are shown in FIGS. 6 and 7. Fluorescence plotted on the vertical axis represents the degraded amount of casein, and that value was estimated to be the value of protease activity. The protease inhibitors added to each sample solution are plotted on the horizontal axis. As a result, Elastase Inhibitor I, EDTA, Leupeptin and TLCK were observed to demonstrate hardly any inhibitory effects against protease present in pancreatic juice. On the other hand, although Aprotinin was observed to demonstrate weak protease inhibitory effects against pancreatin (simulated pancreatic juice), it was observed to demonstrate hardly any effects against the pancreatic juice specimens. In addition, although TPCK was observed to demonstrate slight protease inhibitory effects, since those inhibitory effects did not increase even if concentration was increased, inhibitory effects against protease present in pancreatic juice were judged to be low.

### Example 6

A study was conducted on the protease inhibitory effects of pancreatitis therapeutic drugs consisting of gabexate mesilate (Foy, Wako Pure Chemical Industries), camostat mesilate (Foipan, Wako Pure Chemical Industries) and nafamostat mesilate (Futhan, BD).

More specifically, sample solutions obtained by respectively adding Foy and Foipan at a final concentration of 0.5 mM to 50 mM and Futhan at a final concentration of 0.5 mM to 5 mM were prepared, and the protease activity of each sample solution was measured in the same manner as Example 1 using pancreatin and two types of pancreatic juice specimens. In addition, the protease activities of a sample solution obtained by adding 1 mM PMSF, a sample solution obtained by adding 4 mM AEBSF and a sample solution obtained by adding 5 mM p-APMSF were also measured in the same manner as protease inhibitors.

The results for the protease activity (relative value: %) of each sample solution are shown in FIG. 8. Protease activity based on a value of 100% for the fluorescence intensity of a sample solution to which protease inhibitor was not added is plotted on the vertical axis, and indicates relative values (%) of protease activity estimated from the fluorescence intensity of each sample solution. The protease inhibitors and final concentrations thereof added to each sample solution are plotted on the horizontal axis. On the basis of these results, the protease inhibitory effects of these pancreatitis therapeutic drugs were clearly determined to be lower than those of compounds having a sulfonyl fluoride group.

### Example 7

A study was conducted on the optimum concentrations of PMSF, AEBSF and p-APMSF for inhibiting pancreatic juice protease.

More specifically, sample solutions to which were added various concentrations of PMSF (Roche), AEBSF (Roche) and p-APMSF (Sigma) were prepared for use as protease inhibitors, and the protease activity of each sample solution was measured in the same manner as Example 1 using pancreatin and two types of pancreatic juice specimens. The protease activity of a sample solution to which protease inhibitor was not added (Inhibitor (−)) was also measured in the same manner as a control.

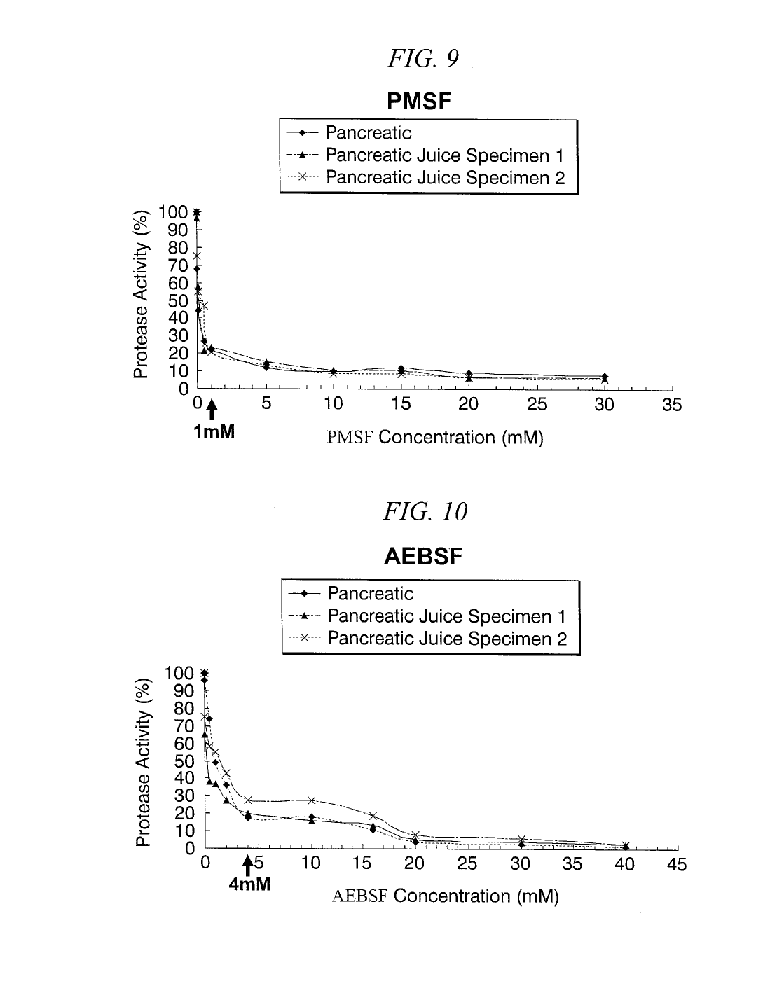

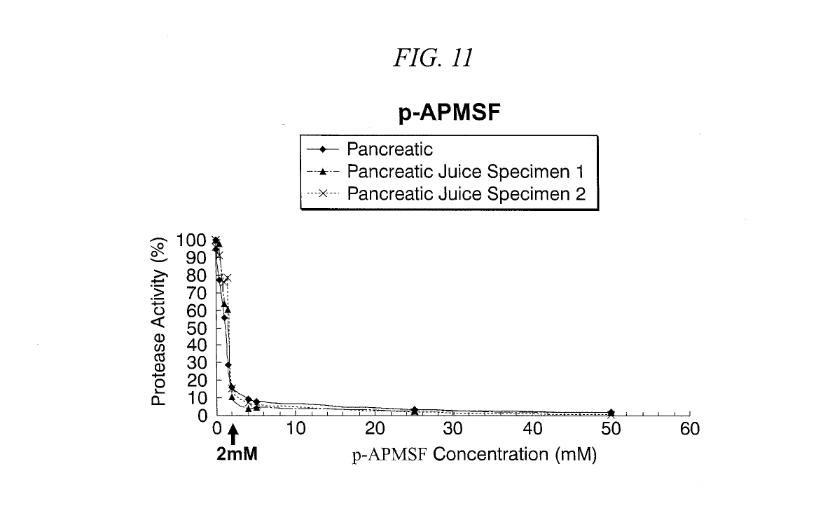

The results for the protease activity (relative value: %) of the sample solution to which PMSF was added are shown in FIG. 9, the results for the protease activity (relative value: %) of the sample solution to which AEBSF was added are shown in FIG. 10, and the results for the protease activity (relative value: %) of the sample solution to which p-APMSF was added are shown in FIG. 11. Protease activity based on a value of 100% for the fluorescence intensity of a sample solution to which protease inhibitor was not added is plotted on the vertical axis, and indicates relative values (%) of protease activity converted from the fluorescence intensity of each sample solution. The final concentrations of the protease inhibitor added to each sample solution are plotted on the horizontal axis. On the basis of these results, high levels of protease inhibitory effects against protease present in pancreatic juice were clearly demonstrated to be obtained in the case of concentration of about 1 mM or higher for PMSF, a concentration of about 4 mM or higher for AEBSF, and a concentration of about 2 mM or higher for p-APMSF.

### Example 8

A study was conducted of the effects on protease inhibitory activity observed as a result of combining PMSF, AEBSF and p-APMSF. PMSF (Roche), AEBSF (Roche) and p-APMSF (Sigma) were used as protease inhibitors.

More specifically, a sample solution obtained by adding 1 mM PMSF, a sample solution obtained by adding 4 mM AEBSF, a sample solution obtained by adding 2 mM p-APMSF, a sample solution obtained by adding 1 mM PMSF and 4 mM AEBSF, a sample solution obtained by adding 4 mM AEBSF and 2 mM p-APMSF, and a sample solution obtained by adding 1 mM PMSF and 2 mM p-APMSF were prepared as protease inhibitors, and the protease activity of each sample solution was measured in the same manner as Example 1 using three types of pancreatic juice specimens and two types of duodenal juice specimens. The protease activity of a sample solution to which protease inhibitor was not added (Inhibitor (−)) was also measured in the same manner as a control.

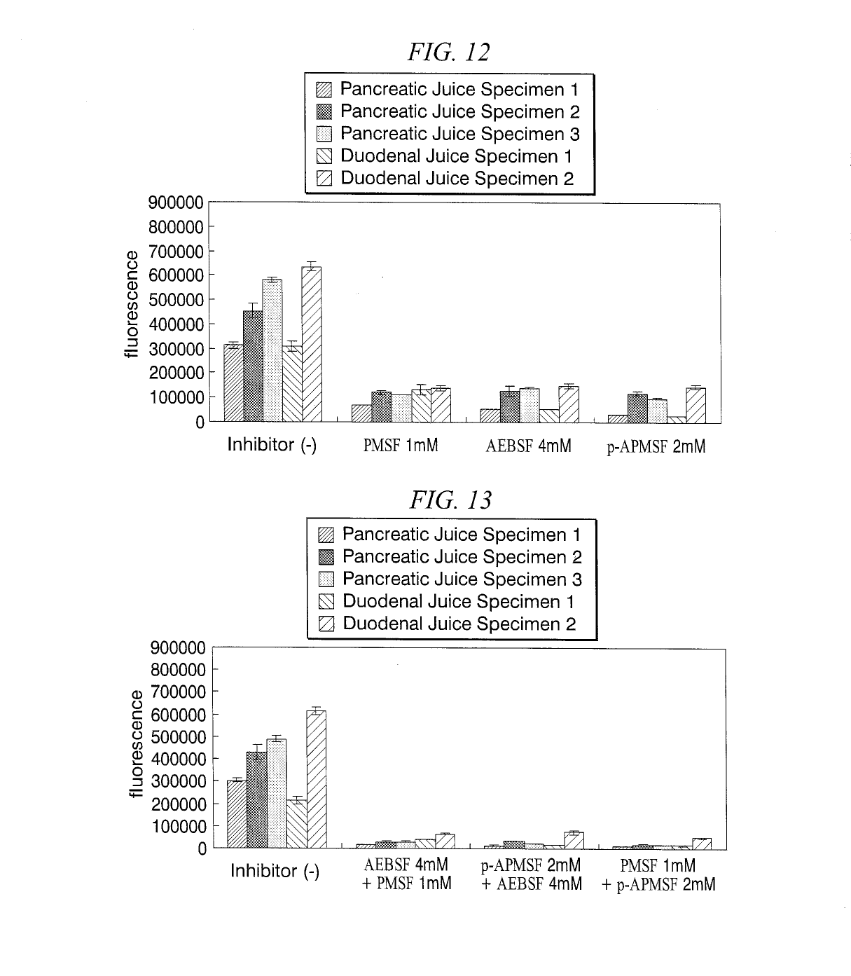

The results of measuring the fluorescence of the sample solutions to which were added one type of protease inhibitor are shown in FIG. 12, and the results of measuring the fluorescence of the sample solutions to which were added two types of protease inhibitors are shown in FIG. 13. Fluorescence plotted on the vertical axis represents the degraded amount of casein, and that value was estimated to be the value of protease activity. The protease inhibitors added to each sample solution are plotted on the horizontal axis. As a result, the case of combining addition of two or more types of protease inhibitors was clearly demonstrated to enhance protease inhibitory effects to a greater degree than in the case of adding one type of protease inhibitor. In addition, regardless of which combination of two types of PMSF, AEBSF and p-APMSF was used, roughly the same high levels of protease inhibitory effects were obtained.

### Example 9

According to the results of the aforementioned Example 8, protease inhibitory effects were clearly demonstrated to be enhanced by the combined addition of two or more types of protease inhibitors selected from the group consisting of PMSF, AEBSF and p-APMSF. Therefore, a study was conducted on whether or not protease inhibitory effects are enhanced by combining two types of these inhibitors with other inhibitors. PMSF (Roche), AEBSF (Roche), Aprotinin (Roche) and TLCK (Sigma) were used as protease inhibitors.

More specifically, a sample solution obtained by adding 1 mM PMSF and 4 mM AEBSF, a sample solution obtained by adding 1 mM PMSF, 4 mM AEBSF and 0.3 mM Aprotinin, and a sample solution obtained by adding 1 mM PMSF, 4 mM AEBSF and 1 mM TLCK were prepared as protease inhibitors, and the protease activity of each sample solution was measured in the same manner as Example 1 using four types of duodenal juice specimens. The protease activity of a sample solution to which protease inhibitor was not added (Inhibitor (−)) was also measured in the same manner as a control.

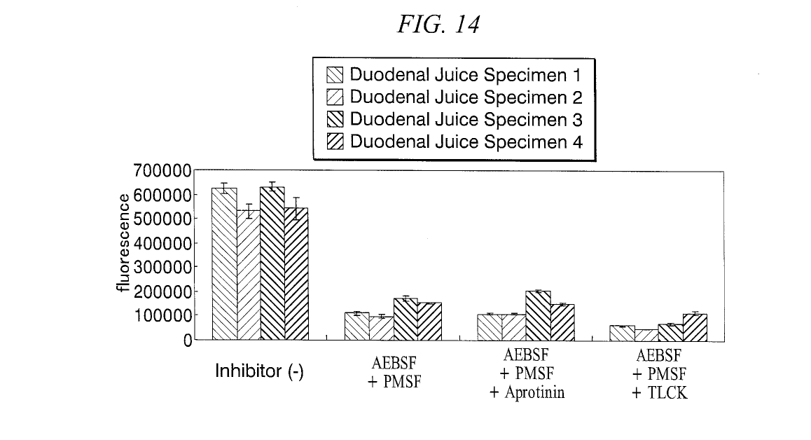

The results of measuring the fluorescence of sample solutions obtained by adding one type of other protease inhibitor not having a sulfonyl fluoride group to PMSF and AEBSF are shown in FIG. 14. Fluorescence plotted on the vertical axis represents the degraded amount of casein, and that value was estimated to be the value of protease activity. The protease inhibitors added to each sample solution are plotted on the horizontal axis. As a result, in the case of having added a peptide-based inhibitor in the form of Aprotinin to two or more types of protease inhibitors having a sulfonyl fluoride group, additional inhibitory effects were not observed in comparison with the case of adding PMSF and AEBSF only. In contrast, in the case of having added a sulfone-based protease inhibitor having a sulfonyl group in the form of TLCK to PMSF and AEBSF, additional inhibitory effects were observed in comparison with the case of adding only PMSF and AEBSF.

The protease inhibitor for a biological sample containing pancreatic juice components as shown in Example, and the protease inhibitory mixture as shown in Example were demonstrated to be able to extremely effectively suppress protease contained in a biological sample containing pancreatic juice components such as pancreatic juice or duodenal juice.

Consequently, the method for inhibiting protease in a biological sample containing pancreatic juice components that uses the protease inhibitor or the protease inhibitory mixture as shown in Example were able to effectively suppress protease activity in a biological sample containing pancreatic juice components. Accordingly, according to this inhibition method, degradation of pancreatic juice-derived components was suppressed, and biological samples containing pancreatic juice components could be stably preserved.

1. A method for inhibiting protease in a biological sample containing pancreatic juice components, comprising:
adding at least one type of protease inhibitor having a sulfonyl fluoride group to the biological sample containing pancreatic juice components, thereby inhibiting protease enzyme activity in the biological sample.

2. The method for inhibiting protease in a biological sample containing pancreatic juice components according to claim 1, wherein the protease inhibitor having a sulfonyl fluoride group is one or more types selected from the group consisting of PMSF, AEBSF, p-APMSF, 4-(fluorosulfonyl)benzoic acid, 3-(fluorosulfonyl)benzoic acid, 2-aminobenzenesulfonyl fluoride, 3-aminobenzenesulfonyl fluoride, 4-aminobenzenesulfonyl fluoride, 2-nitrobenzenesulfonyl fluoride, 3-nitrobenzenesulfonyl fluoride and 4-nitrobenzenesulfonyl fluoride.

3. The method for inhibiting protease in a biological sample containing pancreatic juice components according to claim 1, wherein the protease inhibitor having a sulfonyl fluoride group is one or more types selected from the group consisting of PMSF, AEBSF and p-APMSF.

4. The method for inhibiting protease in a biological sample containing pancreatic juice components according to claim 1, wherein the protease inhibitor having a sulfonyl fluoride group is two or more types selected from the group consisting of PMSF, AEBSF and p-APMSF.

5. The method for inhibiting protease in a biological sample containing pancreatic juice components according to claim 1 wherein PMSF, AEBSF or p-APMSF is added, as protease inhibitor, to the biological sample so that the final concentration of PMSF is 1 mM or higher, the final concentration of AEBSF is 4 mM or higher, and the final concentration of p-APMSF is 2 mM or higher.

6. The method for inhibiting protease in a biological sample containing pancreatic juice components according to claim 1, wherein at least one type of protease inhibitor having a sulfonyl group is further added to the biological sample.

7. The method for inhibiting protease in a biological sample containing pancreatic juice components according to claim 6, wherein the protease inhibitor having a sulfonyl group is an amino acid chloromethyl ketone.

8. The method for inhibiting protease in a biological sample containing pancreatic juice components according to claim 6, wherein the protease inhibitor having a sulfonyl group is one or more types selected from the group consisting of TLCK and TPCK.

9. The method for inhibiting protease in a biological sample containing pancreatic juice components according to claim 6, wherein the protease inhibitor having a sulfonyl group is TLCK.

10. The method for inhibiting protease in a biological sample containing pancreatic juice components according to claim 1, wherein the biological sample is pancreatic juice or duodenal juice.

11. A protease inhibitor for a biological sample containing pancreatic juice components that is a compound comprising a sulfonyl fluoride group, and having protease inhibitory activity; and to be added to the biological sample containing pancreatic juice components for inhibiting protease present in the biological sample.

12. The protease inhibitor for a biological sample containing pancreatic juice components according to claim 11, which is a compound selected from the group consisting of PMSF, AEBSF, p-APMSF, 4-(fluorosulfonyl)benzoic acid, 3-(fluorosulfonyl)benzoic acid, 2-aminobenzenesulfonyl fluoride, 3-aminobenzenesulfonyl fluoride, 4-aminobenzenesulfonyl fluoride, 2-nitrobenzenesulfonyl fluoride, 3-nitrobenzenesulfonyl fluoride and 4-nitrobenzenesulfonyl fluoride.

13. The protease inhibitor for a biological sample containing pancreatic juice components according to claim 11, which is a compound selected from the group consisting of PMSF, AEBSF and p-APMSF.

14. A protease inhibitor mixture comprising at least one type of the protease inhibitor for a biological sample according to claim 12.

15. The protease inhibitor mixture according to claim 14, further comprising at least one type of protease inhibitor having a sulfonyl group.

16. A kit for preserving a biological sample containing pancreatic juice components, comprising at least one type of protease inhibitory having a sulfonyl fluoride group, wherein the kit is used to store a biological sample containing pancreatic juice components.

17. The kit for preserving a biological sample containing pancreatic juice components according to claim 16, further comprising at least one type of protease inhibitor having a sulfonyl group.

18. The kit for preserving a biological sample containing pancreatic juice components according to claim 16,
further comprising a storage container provided with a storage portion for storing collected body fluid, wherein0 the protease inhibitor having a sulfonyl fluoride group is preliminarily contained in the storage portion.
